SEQUENCE LISTING (18 sequences)

SEQ ID NO: 1 - DNA, 12446 bp, Bacillus thuringiensis
  gene (1)..(12446)
  CDS (12015)..(12446)
  CDS (5790)..(7475)
  CDS (2010)..(2315)
  CDS (10957)..(11637)
  CDS (7987)..(8673)
  CDS (4661)..(5278)
  CDS (2617)..(4344)
  CDS (364)..(681)
  CDS (1)..(363)
atgggagtaacttggacttatttcaaacaatttgaaatcgtagaacatgaagaaaatgattttaatgagatgatacgatattttgatcaaggcgaattaagatttacttacgcaacaagtgggacattaagagctgtatacgcaaattatggaatacatattccgatatatagtcaatttgaacctccaaactcaaagaaattagaattagtttctcctgaagatctagttcatgcttgtgaagatgccataaaagttttaaaagaaggaattaacccagaatttaagggttttgatggtgagaaaagtttgctatgggaattggatgatctcgatggacgaaatgggggaagtaaaccatagatggaggaattttatatgtgtatcccaatagtagttctagtagaatatgatttttataatgacggaacaggaagcaaagtctatttattcaaagatatcaataatgcgataatatttgctaaaagagaagcaaaaacatatctagaagataatagccttactctagaagactttaaagggcaacatgatactctagacattatggaaacaaatgatagttattatttcaattcatggaatgataaaaattgtgataaatacaatattgttgtctatgaacaagaatttaaagataaaacaacgaaactaataaactaatcgatcctttctttctctaaagctctctatcactttttttcacctacttttatagaccagctttgatgtaatcaacgaatggtttagcaagtaagaagactactacgccagcaccacagaataaaagaccttttttaaatttaggtgcattttgctcgtcaccttgccaaatcattagaccacaaactaaaccaccaataatgaatgcccaaataaatgtactttgcatgtcagtaaatagcgtttgtaataaatgttgaattcgagtaaacataatttcactccctccttagaataagttagtaagatcagataatacctatttttaaataaaataaccaaaaagggtataaaggattgatttacaaacccaattgcgatcttcaggctaaattatacccttattaccccaaataggcaataagggtataaaaatttttataatttttatttctattaaaaataaaaccattattttctcattaataaggttctatatcagtttcgtaccgaaacctgttgccgtatagtaattcgtccttcccttcatgcaaggggcattaaatttttgaatcattgtgtttcgtttatgctttcacgtcgctccgcaatttgcattgtaagtttagcaaatttctacacttgtaaaagcccaactcaacatcaattctaaaaaatttaatactattcccttgcattccgatcacttctcattacgtataaggcaacaggtaacagagaaaaacctatcaacactactacgaaaagaggaaattttatgttcttatcacaattatctttttatcaattagagattaaaaacacatccccgaaagaagctattacaagctctacaactgaatcattttatgcttatggaagtgcatggttaaaagcctgtaatactattagcaattttttacaacaaaacaattataaaaaagatgatttaaatattgtattcaatgaagacccaaagaatgaagtttatcgttatacttggtcaggaattcacaagtctagtttcaaaaaacttgaaattactattatttatactcaattcgctgatacagaggacttttacagagagtgtacatgctgtaataaagttatgtttgaaggatattgtatacacgagggactagaatatttttgttctgataaatgcttacatacacaatacacgcctgatgaatatgaggaaatgcatgaagatgattatgcttactggacagtatggcttgaataattttgaatttcaattattttattcagaaagagaatcaaaaagaaactcataataaatttatttagaatttattatggatttatataaagttcattttaggtttatttattttttgtaataaagattccattaatgaattcccccactgcgatgcgccgctggcggaagttaaatctgacgaactggaaccactgccggtttcactgaccaatctgaatccgcaaattatccgcgcccgcaccgtgtgcagcggtagtgcaggcggcattctgacgccgatctcttctttagatctcaatgcgctgggtaatcttcccgcagccaaaggcgttgacgccgagcaatccgcactggaaaacggcctgacgctggtactgaaaaacattgagtttcgtctgctggatagcgacggtgctaccagcgcgattctggaagctcaccgatccctggctggcgatacttccctgcgcgaacatttactggcaggcgtcagcgccggattaagctgcgccgaagcaattgttgccagcgcgaatcacttttgcgaagagttttcccgttccagcagcagctacctgcaagaacgtgccctggacgtacgcgacgtctgcttccagttactccagcaaatctacggtgagcaacgcttcccggcaccgggcaaactgacgcagcccgccatttgtatggctgatgaactgacccccagccagttcctcgaactggataaaaatcacctcaaaggattgttgctcaaaagcggcggcaccacctcacatacggtgatccttgcccgttcgttcaacattccaacgctggttggtgtggatattgatgcccttactccgtggcagcaacaaacgatttatatcgacggcaacgccggggcgattgtggttgagccaggggaagccgtagctcgttattatcagcaagaagcccgcgtacaggacgccctgcgtgagcaacagcgtgtctggctgacccaacaagcccgtaccgctgacggtatccgcattgaaattgccgctaacatcgctcactccgtggaagcgcaggccgcattcggcaatggtgcggaaggcgttggtttgttccgcactgaaatgctctatatggatcgcaccagcgcaccgggcgaaagcgagttgtacaacattttttgtcaggcgctggaatccgccaacggacgcagcattattgtgcgcactatggacattggcggcgacaaacccgttgattatctgaacattcccgcagaggcaaacccgttcctcggttatcgcgccgtgcgtatttatgaagagtacgcgtcgttgtttaccacgcagctacggtcgatcctccgcgcctccgctcacggcagcctgaaaatcatgatcccgatgatctcctcaatggaagagatcttatgggtgaaagaaaaactggcggaagccaaacagcaactacgtaacgaacacattccgtttgatgagaaaatccagctcggcatcatgctggaagtgccgtcggtgatgttcatcatcgatcaatgctgcgaagagattgatttctttagtattggtagtaatgacctgacgcagtatctgctggcggtggatcgcgataacgctaaggttactcgtcactacaacagcctgaatccggcattcttgcgggcgctcgattacgccgtgcaagcggtgcatcgccagggcaaatggattggtctgtgcggtgagctgggagcgaaaggttccgtgctgccgttgctggtcggcttagggctggatgaactcagcatgagcgcaccatcaattccggcggcgaaagctcggatggcgcaacttgatagccgtgagtgccgcaagttgctcaaccaggcaatggcctgccgtacttcgctggaagtagaacacctgctggcgcaattccgcatgacccaacaagacgcaccgctggtcaccgccgagtgcatcacactggaaagcgactggcgcagcaaagaagaagtgctcaaaggcatgaccgataacctgctgctggcgggccgctgccgctatccgcgtaaactggaagccgacttgtgggcgcgcgaggccgttttctctaccggtctgggctttagttttgccattccacacagcaaatcagaacacattgagcaatccaccatcagcgtggcgcgtctgcaagcgccggtgcgctggggcgatgatgaagcgcaattcatcattatgttaaccctgaacaaacacgctgcgggcgatcagcatatgcgcattttctcgcgcctcgctcgccgcatcatgcacgaagaattcgagctcggtacccggggatcctctagagtcgaccaagagaaacagtatgttaccttatatttttggaaattgaaaacaggttattattgtagctatcacaaatactaaaaagttaaatcaatcaataatagacaacactaactctagtattatattaatttatgattaatcaacgctgactgtatttccttgagactctaataagacaggtttattattaatatagaaaataaagttggatttaaatactataaaaaatatatagaattaaggagattgggatataaaacaaaaagatattgagggtgtgcttattgtgaaaattacaatagcaggaataggatacgtaggactatctaatgcgattttattgtctcaaaataacgaagttatagcatttgatattattcaaaaaaaagtagatatgataaatgataaaaaatctcctattttagatgatgaaattgagaaatttttagcaacaaaagaattaaatttaattgcaactaccgatagttacaaagcatttaaggatgctgattatttaattattgcaacgccaacagattatgatccagaaaaaaattcttttaatacaaggaccgttgaaactgtaattgctaaaatcttaacaattaatccagaagctataatgataattaaatcaactgtacccgtaggctatacagaaaaagtaaagcgaaagtttaaaacaagtaatattattttttcaccagaatttttaagagagggtaacgctttatatgataacttatatccttcacgtataatcgttggtgaacagtctagtagggctaaagtatttgctgacttgttagtagaaggagctgctaagaaagatattcctgtcttatttacaaactcaactgaagcagaagcgattaaattgtttgctaacacatatttagcaatgagagtggctttttttaatgaattagattcatatgcagaagtaagaggattacacacaaaacaaattatcgatattaaggggaagttcaattttaccttccccttttaaatattcagataaaaaatatagcgctcgtccctcaacaaatagttccaatgggccttttactgataggcagcgattatcagaatcgaaagtaacactagtaatttcatcttgaatgtaatttattaatcccgatattttaattgcttctacttggttatgtgtattcaatcccaattctttttgaatcgatgactcaaatttaggaattcttttgatagggggagatggagtaacagctggatatacagatgaatccttaaataaagagaggtttgtagtaattaatggggaaaaatattacaatactggcgatgtagtttcatgtaataaagatcaactatactaccatggaagaaatgatagtcaaatacaaattaacggtatacgtgtagaattaggtgaaattgaatatttacttgaaaaaatacatggggttcaacaagcggttgttttattttatcaggataaattgcttgcatttatcctgagttctaatttaacaattcatgatatgaaaaaaatggctacttcagtacttgaaaggtatatgtttccaaatgattatcatatcatcaagacgtttccattgaccgaaaataataaagttgaccgaaaatctttattattttcttatgaagagagtaagaaatcggaaaaactaatcattagtaaggatatgacagaatttgaagaggttttaagagataaagtcgctctaatacttaatttaccaaaagagttaattggaaaagatagtgatttctttgagttaggtggagattccctagatgtatttcaattacttttaaaattagaagaaatgtatgaaacagagctttctttggagttaatttatactaatcgaacattatctaggattgcttccgaagtttcaaaattattaacaacagaaagagaagaagtttttgaagaaaagctacatgaggaagactttaagctcatggaaaaagaaatcaacggttatttattaaatgatgaatatcaaactacttatagctttgaaactatacactcacaacgggtttactattttgataattttaagagttcagtttcctttgattatagggtagatacttcatacgataaggaaacagtaataaaagcaattaaaactattattaatagcaatgatttgatgagagctatgttaagggaaagtgattccagattagaatttaaaattttaaattccattgatagttttcctatatttacattaaatcgaaatgttgaaaaaaaatcatttatccaaaaaatcgaatcaataggagaaaacttggtgtataaggctcgcaataatggtgggttactcggttttttagccttactaatagataaatcagggtatacaatagttggtgtgttagatcatactattgctgatatctcatgtattaatataattaaaaaaatgattggtgaggaactaaatggctattcaagtaaagaaaaggcaagttataatgatttctgttatgaggttagagaatataatacaattgatagcttaaaaaataatgcatatcataataatttacttgctataagcaaaaatgtgaatgatgtcaacttaaataagctaagtaaaagtttaaaatgttatgaaatagattatcctagccaaggtgattcttttcggattataaattttctttcatatgttataggaaaaagattattagaaatcgtagatcgagaacaaattgtaataaaatcagtcattaacgggcgagaaaataagctttttgatttttcaaccacaatcggtgattttcatggtagtttatatttaatttattcaaaagatgaaaactatgaacaatttacgaataaatctgaggggatattcggaaaatactacactacccacccatacagaccagggtatgcgtttggttcaaattacccaagtaaaacaaaagaacaaaaaaaacttaaagatcagtggaattcgatttcaaatacctcaataaattatattggagaagtatcagattacgaaaagagctcttattatgatacaatcagcaatgtctttgataacttggaaaaaatacaagatttaatttatgttacagcttttagtaataataataaattaacggtattcttaaataaaaatttaggccaactaaatttccttgaaaaattgtactatccaactttcatttaaatcatgaattgttaaattagaactcaggataaatgcaagcaatttatcctgataaaataaaacaaccgcttgttgaaccccatgtattttttcaagtaaatattcaatttcacctaattctacacgtataccgttaatttgtatttgactatcatttcttccatggtagtatagttgatctttattacatgaaactacatcgccagtattgtaatatttttccccattaattactacaaacctctctttatttaaggattcatctgtatatccagctgttactccatctccccctatcaaaagaattcctaaatttgagtcatcgattcaaaaagaattgggattgaatacacataaccaagtagaagcaattaaaatatcgggattaataaattacattcaagatgaaattactagtgttactttcgattctgataatcgctgcctatcagtaaaaggcccattggaactatttgttgagggacgagcgctatattttttatctgaatatatgtctgaatgcttacaggcttcctacacaaatagtttattgatacatgcggcagccgtttattcaaaagaaaaagatagatcatatttgattttaggagaaaaagggtctggtaaaacaacattgtcctttagattatgtcaagaattaggattaagtcttattggtaatgacttagttcgtattggatatgatgaaaatggtgaactttttacaaaagaaggaagtcgatggttcgatgtaagagagacagcagtaaaggctgacgattatatgaataagttagctactatattatctgctaaatcggcaaattcatggaataataaaactagaatattaccagaagatcattctatagaaacccattttgagcaatcgaaaatagataaaattttaaatattagaattgatccttatcaaaattatttttctgtttctccatgggaaggggtacagagaaatttaatacttcatgaaaaaataggtaggcatatatctggtcaggctacaccatttcaagatgatcagggtaattatcttggatcactaccaagcataaatcgtgataaggcttcattggtaagggataatatagttaagtgtatggtcaatacaggaataactgaactatttggtccggacagtaaagcacttgcaacctggtttaaggaagaagtactatgattgctatcggaattccaacttataatgaagcaaaaaatatttctaaacttactcaattaattgattgtgtagcagtaaaaatagggctagaaatagttattattaatgcagacaataacagtccagataatacggccacaatatttacatcagttaaaacattaaataaaaaaatatctgttgttaccaaggaagttggtaaaggtttcaatattaaagcaataattgatattgtaaataacctagataattgtgaaggttgcatattaattgatggtgatattacatctttttcagagtcttggcttaaaaaatttattgaacttttaatagacaaagtagattttgttgtacctaattattcaaggagttttcaggaagggaacacaacaaatcactttgtttatccattaatcaattaccatacgaacggcaattgcccaagacagccaatcgctggtgattttggtttatcgaaaaatttcatcaaatttcttgtcaattctgtgcactggcacaaatattgttatggctatggtattgatatatttcttactttacatgctgtatataataattttcgaatagaggagatcaatctagatagaaaagaacataaccctagtttcgggaaaatggtagacatgtttgttgaagttgcgagtagttattatgaaacctccaaaatcatatttgaaagtaaaaagaaaaatggcattttcaatacagcagtaataaaatctagtaatccatcagttttattttctcctaaaatatttttaagccaagaggaaatattaaagaaaaaagaggaagctaataaaatattagcatcgaaagagacaatattaaatatgagaaaagatattgaaattaatccatcagtttgggtggaaatattgcttgctcatgagaaaaggataggacaggtagatagtcaattgcttgcaaaatcaatattgccgtattatttgttacgtgttgtagactatttacaaaacatttctaatgtgaaagatgcagaacttaatatcgaaaaggaaattgatttattgcaattacaaaaaaatgaagctgtcaaatcagatgttgaatttgatgtagattttataaacagtatttaaattaaagattaataccttgtattttttaaaaattcgaataactatccttttccttgaaaacttcatcatacggactatttgactatcttattgtaagatccaaatagtccgttcatcactttaaaatgataacattaaaaatttgaattatagtgcaaatggtttataattcttattctattttaatggcttctatttgttgaaaaatggagtaatttctctatttcaaattaatttagagatgaattatgaagcttaaatttttcgatatcggtatagtccctccttcgcttgtttgttgtatcaaagctaaatgaactattgggaatgatgaggagaataaatattgaaattatttttaagttggtaggagggaacataatggatcgttcaaagaaaaatggtatatatatgcttacgggtaaaacaacaaataaaataggtaatgtaatttttgattatataaataatgtattaatagcttctttaggaacaaaggcttctaaccttttagcactatatcaaagttctgaaattattgtaaatatattgtttaatatgttaggtggtgtttttgcagatttaaaaaataggaagaaaataattattataacagatttaactgcttcactagcaacctttctactctatcttttttgggatagtaaaaattccttatttttaattattttaataaacattttattggcgttattgtattcttttaattcccctgcatataaagctattgttaaagatatactgaaaacagaagatttaaataaattcaattcatactctaatgctttttcagaagtggtttctgtattgggtcctttagttgctataagtaccgtacattattttggatttaagacaggaatgttaattaatagtattagttttcttatttctgcttattgtgtaagtagatttcaagttataaattatgtgcagaaaaagaaaaagggtaaagaaaaatatatggtagttctttcagatggttttaaatatgttttgaagcagcgtaatgttcttgaattattaattgtatcttcttttattaatttttttctagcaggatataatttctttttaccatatacaaatattttttctgaaaatcaaggtatctatgcatccattttaattacagaatctatagggagtattgttggggctcttttaaatcgataccgtcgacctcgagggggtgcagcaggtgataaatatcctgtttgccgtcccagactttggtggtgaccttattgtccgcatccagctcctgccaccaggaaccattttcatagtccatcaggtacttaatgcagtactcccaccatgtttgataccaggtttcatactggcgatcgccagtgacggtgtagagcgcgtaggccgtacccattgcttcgacgataggccaacgtacacgttcgcggaccaccggttttccttcccagtcaacggtataaacaatcccgtccgcgccatcgggtgcccaggcatcgcgcacggtggcgttaaacagacctttggcatcttctagcagccatgctggtggttgttcgcaacgggcttccagggcagcatggatgtgcagcattaaacggccccattcgatccagtggcctggtgtacctccgaacgcgcggaagcgatgcgccgggttatctttgttgtaatccggcagcggattccactgggtatcgaagtgttcgttaacgcgataatgattatttctggcgacgtcgtggataatcacggaagccacgcgaatcgcgcgatccagccattttttgttgtgagggacgtcataaacaatcaagaaagcttgcattgtgtggtgacggtcttccagccattcggctcctgctgtattgaagcataccaggctatttcaatatcgctatgctgcggcagcatttaaccccttgtaattcattgccataattgatttaattcacaaataaaactataacatggtgaaattaataaaaaaacacagatgatggggctagaatttacacaccacttaccctaaagctttatgactggtgggttttgggagtatcaaatcggcttgcatggggatgtcctacaaaggaacaccttcttccacactttctggaacatttaggtaacaaccatctggatattggtgttggaactgggttttaccttactcacgtacctgagagtagtctgatatctttaatggatttgaacgaagctagcctgaacgcggcatctacaagggctggggaatcaaaaattaaacataaaattagccatgatgtttttgaaccttatcccgcggcgttacatggtcaatttgattccatttccatgtcttaccttcttcactgcctgcctggaaatatatctacaaaaagctgtgtaatacgcaatgcggcgcaggccttaactgacgatggaactctatacggagccacaattcttggcgatggagttgtgcacaatagcttcggtcaaaaactgatgcgcatttacaatcagaaaggcatcttttcaaacacaaaagattccgaagaaggcttaacacatatactctcagagcatttcgagaatgttaaaaccaaggttcaaggtactgtagtaatgttttccgcttcaggaaaaaaatag

SEQ ID NO: 2 - protein, 120 aa, Bacillus thuringiensis
MGVTWTYFKQFEIVEHEENDFNEMIRYFDQGELRFTYATSGTLRAVYANYGIHIPIYSQFEPPNSKKLELVSPEDLVHACEDAIKVLKEGINPEFKGFDGEKSLLWELDDLDGRNGGSKP

SEQ ID NO: 3 - protein, 105 aa, Bacillus thuringiensis
MEEFYMCIPIVVLVEYDFYNDGTGSKVYLFKDINNAIIFAKREAKTYLEDNSLTLEDFKGQHDTLDIMETNDSYYFNSWNDKNCDKYNIVVYEQEFKDKTTKLIN

SEQ ID NO: 4 - protein, 12 aa, Bacillus thuringiensis
LYKVHFRFIYFL

SEQ ID NO: 5 - protein, 11 aa, Bacillus thuringiensis
IPPLRCAAGGS

SEQ ID NO: 6 - protein, 25 aa, Bacillus thuringiensis
RTGTTAGFTDQSESANYPRPHRVQR

SEQ ID NO: 7 - protein, 17 aa, Bacillus thuringiensis
CRRHSDADLFFRSQCAG

SEQ ID NO: 8 - protein, 7 aa, Bacillus thuringiensis
SSRSQRR

SEQ ID NO: 9 - protein, 17 aa, Bacillus thuringiensis
RRAIRTGKRPDAGTEKH

SEQ ID NO: 10 - protein, 575 aa, Bacillus thuringiensis
MADELTPSQFLELDKNHLKGLLLKSGGTTSHTVILARSFNIPTLVGVDIDALTPWQQQTIYIDGNAGAIVVEPGEAVARYYQQEARVQDALREQQRVWLTQQARTADGIRIEIAANIAHSVEAQAAFGNGAEGVGLFRTEMLYMDRTSAPGESELYNIFCQALESANGRSIIVRTMDIGGDKPVDYLNIPAEANPFLGYRAVRIYEEYASLFTTQLRSILRASAHGSLKIMIPMISSMEEILWVKEKLAEAKQQLRNEHIPFDEKIQLGIMLEVPSVMFIIDQCCEEIDFFSIGSNDLTQYLLAVDRDNAKVTRHYNSLNPAFLRALDYAVQAVHRQGKWIGLCGELGAKGSVLPLLVGLGLDELSMSAPSIPAAKARMAQLDSRECRKLLNQAMACRTSLEVEHLLAQFRMTQQDAPLVTAECITLESDWRSKEEVLKGMTDNLLLAGRCRYPRKLEADLWAREAVFSTGLGFSFAIPHSKSEHIEQSTISVARLQAPVRWGDDEAQFIIMLTLNKHAAGDQHMRIFSRLARRIMHEEFELGTRGSSRVDQEKQYVTLYFWKLKTGYYCSYHKY

SEQ ID NO: 11 - protein, 205 aa, Bacillus thuringiensis
MINDKKSPILDDEIEKFLATKELNLIATTDSYKAFKDADYLIIATPTDYDPEKNSFNTRTVETVIAKILTINPEAIMIIKSTVPVGYTEKVKRKFKTSNIIFSPEFLREGNALYDNLYPSRIIVGEQSSRAKVFADLLVEGAAKKDIPVLFTNSTEAEAIKLFANTYLAMRVAFFNELDSYAEVRGLHTKQIIDIKGKFNFTFPF

SEQ ID NO: 12 - protein, 561 aa, Bacillus thuringiensis
MKKMATSVLERYMFPNDYHIIKTFPLTENNKVDRKSLLFSYEESKKSEKLIISKDMTEFEEVLRDKVALILNLPKELIGKDSDFFELGGDSLDVFQLLLKLEEMYETELSLELIYTNRTLSRIASEVSKLLTTEREEVFEEKLHEEDFKLMEKEINGYLLNDEYQTTYSFETIHSQRVYYFDNFKSSVSFDYRVDTSYDKETVIKAIKTIINSNDLMRAMLRESDSRLEFKILNSIDSFPIFTLNRNVEKKSFIQKIESIGENLVYKARNNGGLLGFLALLIDKSGYTIVGVLDHTIADISCINIIKKMIGEELNGYSSKEKASYNDFCYEVREYNTIDSLKNNAYHNNLLAISKNVNDVNLNKLSKSLKCYEIDYPSQGDSFRIINFLSYVIGKRLLEIVDREQIVIKSVINGRENKLFDFSTTIGDFHGSLYLIYSKDENYEQFTNKSEGIFGKYYTTHPYRPGYAFGSNYPSKTKEQKKLKDQWNSISNTSINYIGEVSDYEKSSYYDTISNVFDNLEKIQDLIYVTAFSNNNKLTVFLNKNLGQLNFLEKLYYPTFI

SEQ ID NO: 13 - protein, 228 aa, Bacillus thuringiensis
MSECLQASYTNSLLIHAAAVYSKEKDRSYLILGEKGSGKTTLSFRLCQELGLSLIGNDLVRIGYDENGELFTKEGSRWFDVRETAVKADDYMNKLATILSAKSANSWNNKTRILPEDHSIETHFEQSKIDKILNIRIDPYQNYFSVSPWEGVQRNLILHEKIGRHISGQATPFQDDQGNYLGSLPSINRDKASLVRDNIVKCMVNTGITELFGPDSKALATWFKEEVL

SEQ ID NO: 14 - protein, 7 aa, Bacillus thuringiensis
GVLLGLF

SEQ ID NO: 15 - protein, 119 aa, Bacillus thuringiensis
IDTVDLEGVQQVINILFAVPDFGGDLIVRIQLLPPGTIFIVHQVLNAVLPPCLIPGFILAIASDGVERVGRTHCFDDRPTYTFADHRFSFPVNGINNPVRAIGCPGIAHGGVKQTFGIF

SEQ ID NO: 16 - protein, 19 aa, Bacillus thuringiensis
QPCWWLFATGFQGSMDVQH

SEQ ID NO: 17 - protein, 77 aa, Bacillus thuringiensis
TAPFDPVAWCTSERAEAMRRVIFVVIRQRIPLGIEVFVNAIMIISGDVVDNHGSHANRAIQPFFVVRDVINNQESLH

SEQ ID NO: 18 - protein, 143 aa, Bacillus thuringiensis
MDLNEASLNAASTRAGESKIKHKISHDVFEPYPAALHGQFDSISMSYLLHCLPGNISTKSCVIRNAAQALTDDGTLYGATILGDGVVHNSFGQKLMRIYNQKGIFSNTKDSEEGLTHILSEHFENVKTKVQGTVVMFSASGKK

(19) United States
(12) Patent Application Publication
(10) Pub. No.: US 2012/0015404 A1
(43) Pub. Date: Jan. 19, 2012

(54) GENE CLUSTER FOR THURINGIENSIN SYNTHESIS

(75) Inventors: Ming Sun, Wuhan Hubei (CN); Ziniu Yu, Wuhan Hubei (CN); Lifang Ruan, Wuhan Hubei (CN); Shouwen Chen, Wuhan Hubei (CN); Xiaoyan Liu, Wuhan Hubei (CN); Donghai Peng, Wuhan Hubei (CN)

(73) Assignee: HUAZHONG AGRICULTURAL UNIVERSITY, Wuhan Hubei (CN)

(21) Appl. No.: 13/145,071

(22) PCT Filed: Dec. 15, 2009
(86) PCT No.: PCT/CN09/01446

§ 371 Date: Jul. 18, 2011

(30) Foreign Application Priority Data
Jan. 19, 2009 (CN) 200910060530.4

(51) Int. Cl. C12P 19/44; C12N 1/20; C12N 15/61

(52) U.S. Cl. 435/74; 536/23.2; 435/252.5

(57) ABSTRACT

The invention belongs to the technical field of genetic engineering of agricultural microorganisms. Specifically, the invention relates to producing and using gene cluster for thuringiensin synthesis from Bacillus thuringiensis. The gene cluster includes 11 genes of thuA, thuB, thuC, thuD, thuE, thuF, thuG, thu1, thu2, thu3 and thu4. These genes have the nucleotide sequence as shown in SEQ ID NO: 1. And the thuringiensin biosynthetase encoded by these genes have the amino acid sequences as shown in SEQ ID NO: 2-12. The genes according to the invention and the proteins encoded by those genes can be used as new nucleosides lead compound of pesticide library and provide new target for developing insecticides. In practical use, a series of new, efficient and low toxic insecticide can be obtained by structure modification.

### TECHNICAL FIELD

The invention relates to a nucleotides insecticidal toxin and the gene encoding the toxin, particularly, it relates to a gene cluster for synthesizing (encoding) thuringiensin, and the gene cluster is originated from Bacillus thuringiensis CT-43 strain. The invention belongs to the technical field of genetic engineering of agricultural microorganisms.

### BACKGROUND ART

Bacillus thuringiensis is a kind of gram-positive bacterium widely present in soil. The main characteristics of Bacillus thuringiensis different from Bacillus cereus and Bacillus anthracis are: when the spores are formed, one, two or even more parasporal crystals with protein properties in a shape of diamond, square or irregular shape are also formed inside one end or two ends of the bacteria, and the parasporal crystals are consisted of insecticidal crystal proteins (ICPs) and account for about 25-30% of the cell based on dry weight (Study and Application of Insecticidal Crystal Protein and the Gene Thereof from Bacillus thuringiensis, by YU Ziniu; Research Advances of Several Fields in Bioscience and Soil Science, editor in chief by LI Fudi; page 170-179; China Agriculture Press, 1993, Beijing). Crystal proteins have poisonous activity on the harmful targets such as 500 species of insects in 9 orders of Arthropoda including Lepidoptera, Diptera, Coleoptera, etc, animal and plant parasitic nematodes in Nemathelminthes, trematodes in Platyhelminth, plasmodium and trichomonas in Protozoa, and cancer cells, and the like; however the crystal proteins have no poisonous activity on vertebrates (Schnepf E, Bacillus thuringiensis and its pesticidal crystal proteins. Microbiol. Mol Biol Rev, 1998, 62: 775-806; Pigott C R, Role of receptors in Bacillus thuringiensis crystal toxin activity. Microbiol. Mol Biol Rev, 2007, 71: 255-281). At present, Bacillus thuringiensis is the only insecticide which has the maximum yield, is most widely used and capable of competing with chemical pesticides in the world, it has higher insecticidal efficiency with no poisonous on human and animals, no pollution on environment, and no harm to natural enemies of pests and beneficial creatures; Bacillus thuringiensis enables to maintain ecological balance, and pests are difficult to generate drug-resistance on it; Bacillus thuringiensis plays an important role in sustainable control of pests in agriculture and forestry, and it is widely used to control agricultural pests, forest pests, storage pests and medical insects.

Thuringiensin is a kind of insecticidal active substance with low molecular weight (701 kDa) produced by certain varieties of Bacillus thuringiensis, it has the properties of broad spectrum, low toxicity, good thermostability and slow degradation, etc. Thuringiensin, also called β-exotoxin, fly-killing toxin or thermostable exotoxin, is nucleotides substance which consists of adenosine, ribose, glucose, allomucic acid and phosphoric acid in a molecular ratio of 1:1:1:1. Due to structural differences, thuringiensin has a plurality of analogues. Thuringiensin is a DNA-dependent inhibitor of RNA polymerase, which competes for enzyme binding site with ATP so as to cause death of insects.

At the earliest, thuringiensin was found to be produced from subspecies of Bacillus thuringiensis at the stage of vegetative growth reaching to the highest peak in 24 hours. At present, it is known that thuringiensin mainly exists in Bacillus thuringiensis serotype H1 strain. It is reported that the serotypes which can generate thuringiensin also include =H4ac, H5, H7, and H8 to H12 with a total of 8. Recently, with the further development of purifying methods and toxicology tests, it was found that purified thuringiensin itself has no mutagenicity, indicating that thuringiensin will be a new insecticidal element with great potential.

Based on research, thuringiensin has broad spectrum insecticidal activity with different degrees of the activity on Lepidoptera, Diptera, Orthoptera, Coleoptera, Hymenoptera, Hemiptera, Isoptera, etc, and it is also reported that it has insecticidal activity on aphids, nematodes and mites. When the larvae of housefly take in thuringiensin, the half lethal dose is 0.5 μg/ml; when the larvae of Galleria mellonella were injected with thuringiensin, the half lethal dose is 0.005 μg/g. Compared with several common pesticides, the toxicity of thuringiensin is 1000 times of that of DDT on the injected larvae of Galleria mellonella; however, its the toxicity on Aedes aegypti is less than E605; the activity of thuringiensin on three kinds of phytophagous insects including Phaedon cochleariae, Plutella maculipennis and Pieris brassicae is more than E605, having basic activity similar to commercial agents. Toledo et al. (Toledo J, Toxicity of Bacillus thuringiensis beta-exotoxin to three species of fruit flies (Diptera: Tephritidae). J Econ Entomol 1999, 92:1052-6) found that the LC₅₀ values of thuringiensin on the third stage larvae of 3 kinds of fruit flies (Anastrepha ludens, A. obliqua and A. serpentine) are respectively 0.641, 0.512 and 0.408 μg/cm². Tsuchiya et al. (Tsuchiya S, Assessment of the efficacy of Japanese Bacillus thuringiensis isolates against the cigarette beetle, Lasioderma serricorne (Coleoptera: Anobiidae). Invertebr Pathol, 2002, 81: 122-126) found that, 28 strains among 2652 strains of Bacillus thuringiensis isolated in Japan have insecticidal activity on cigarette beetle; with further study it is found that the insecticidal active substance exists in the supernatant of the liquid culture, and the insecticidal activity was still completely kept after treating at 100° C. for 10 min. ZHANG Jihong et al. (ZHANG Jihong et al., Comparison of toxicity and deterrence among δ endotoxin, spore and thuringiensin A of Bacillus thuringiensis against Helicoverpa armigera (Hubner), Acta Entomologica Sinica, 2000, 43: 85-91) found that, although low concentration of thuringiensin A has no acute lethal effect on Helicoverpa armigera, it has unrecoverable inhibiting effect on growth and development of Helicoverpa armigera, even at the lowest testing concentration the weight of the neonate larvae have no apparent increase in 12 days. In 1970s and 1980s, Bacillus thuringiensis preparations containing thuringiensin were considered as broad spectrum insecticidal elements in the world. At present, Bacillus thuringiensis preparations containing thuringiensin are still widely used in districts of the former Soviet Union, in 1999 a bacterial insecticide for controlling pests including aphids and red spiders (i.e. Bacillus with two toxins) was developed by Russian Academy of Sciences.

With the improvement of living standards, the demands of pollution-free agricultural products and sideline products, green products are also increasing. Additionally, the trade barrier in the developed countries including Europe and America also require promoting the application and popularization of pollution-free green pesticides. Therefore, developing green and environment-protecting agriculture is an important way to improve the export of agricultural products. Biological control is the foundation of developing green and environment-protecting agriculture, and microbial pesticides as important tools for biological control have the characteristics of harmless to human, animals and natural enemies of pests, they do not pollute the environment, can maintain the ecological balance and keep the sustainable development of agriculture and forestry. Among the direct use of biogenic pesticides, antibiotic insecticides play very important roles. In the international market, abamectin and spinosad are respectively the first and third microbial pesticides of big output value. As a new insecticidal element, thuringiensin has the excellent properties such as broad spectrum, low toxicity and good thermostability, and it will have broad market prospects. Since the middle of 1990s, the technology of producing abamectin and the products are introduced into China, the production and application of abamectin developed very rapidly, and only its output value in 2001 reached 580 millions RMB. As the popularization of using abamectin, the drug resistance of pests is also increasing rapidly, and there is an urgent need to develop desirable alternative insecticide.

Furthermore, the successful development of thuringiensin is of important significance. Theoretically, it will add new nucleosides lead compound to the pesticide library and provide new target for developing insecticides; in practical use, a series of new, efficient and low toxic insecticides can be obtained by structure modification, thus adding new alternative pesticide varieties for the high-toxic pesticides in China.

At present, researches on thuringiensin are mainly focused on increasing of fermentation titer, recovering, purifying and toxicity tests. Espinasse et al. (Espinasse S, Correspondence of high levels of beta-exotoxin I and the presence of cry1B in Bacillus thuringiensis. Appl Environ Microbiol, 2002, 68:4182-6) found that the cry1B and vip2 genes carried by a strain were connected to the high yield of thuringiensin by the strain. GAO Suisheng et al. obtained UV1-3 strain with a high yield of thuringiensin by UV-induced mutagenesis, and the yield of the strain was 1.73 times of that of the starting strain B. t subs p. darmstadiensis HD199. Tzeng et al (Tzeng Y M, Penicillin-G enhanced production of thuringiensin by Bacillus thuringiensis subsp. darmstadiensis. Biotechnol Prog, 1995, 11:231-4) investigated the effect of penicillin G on the thuringiensin yield of HD199 strain, indicating that penicillin G can promote cell to release thuringiensin, and fermentation unit of thuringiensin may reach 2600 mg/L, and the expression level was increased by 2-10 times. Huang et al. (Huang T K, Cultivation of Bacillus thuringiensis in an airlift reactor with wire mesh draft tubes. Biochem Eng J 2001, 7:35-39) produced thuringiensin in a airlift reactor with two metal-mesh ventilating pipes, and the thuringiensin yield was increased by 70% than the traditional culturing method by controlling the ventilation and using defoamer in the production process. Wu et al. (Wu W T, Effect of shear stress on cultivation of Bacillus thuringiensis for thuringiensin production, Appl Microbiol Biotechnol, 2002, 58:175-177) cultured Bacillus thuringiensis with tower bio-reactor, and the fermentation unit was increased by 43% through adjusting the stirring and ventilating state. It can be seen that, the thuringiensin yield can be greatly increased through the measures such as widely screening the strains, induced mutation breeding and improving the fermentation process, those are main measures for increasing the strain's capability of producing thuringiensin at present.

So far, there is no report on research of gene cluster encoding thuringiensin in China or abroad.

### Contents of the Invention

The object of the invention is to overcome the deficiency of the prior art, and obtain a gene cluster for synthesizing (or encoding, “synthesizing” and “encoding” have the same meaning in the invention, similarly hereinafter) thuringiensin, the gene cluster is originated from Bacillus thuringiensis CT-43 strain and can synthesize (or encode) the thuringiensin which has broad-spectrum insecticidal activity with different degrees of the activity on Lepidoptera, Diptera, Orthoptera, Coleoptera, Hymenoptera, Hemiptera, Isoptera etc, and also having high insecticidal activity on aphids, nematodes and mites, particularly nematodes.

The present invention provides a gene cluster from Bacillus thuringiensis CT-43 strain encoding biosynthetase capable of synthesizing thuringiensin, which comprises 11 genes of thuA, thuB, thuC, thuD, thuE, thuF, thuG, thu1, thu2, thu3 and thu4 with the nucleotide sequence as shown in SEQ ID NO: 1. The positions of the genes in the cluster respectively are as follows: thuA locates in No. 1-363 bases of the nucleotide sequence of the gene cluster with a length of 363 base-pairs encoding 120 amino acids and encoding glucose-6-phosphate dehydrogenase (G-6-P-DH); thuB locates in No. 364-681 bases of the nucleotide sequence of the gene cluster with a length of 317 base-pairs encoding 105 amino acids and encoding helicase; thuC locates in No. 2617-4344 bases of the nucleotide sequence of the gene cluster with a length of 1727 base-pairs encoding 575 amino acids and encoding phosphate transferase; thuD locates in No. 4661-5278 bases of the nucleotide sequence of the gene cluster with a length of 617 base-pairs encoding 205 amino acids and encoding UDP-glucose dehydrogenase; thuE locates in No. 7987-8673 bases of the nucleotide sequence of the gene cluster with a length of 686 base-pairs encoding 228 amino acids and encoding Shikimate kinase (SK); thuF locates in No. 8670-9797 bases of the nucleotide sequence of the gene cluster with a length of 1127 base-pairs encoding 375 amino acids and encoding glucosyltransferase; thuG locates in No. 10957-11637 bases of the nucleotide sequence of the gene cluster with a length of 680 base-pairs encoding 226 amino acids and encoding glucosamine isomerase. 2 dehydrogenase genes, 1 helicase gene, 1 phosphate transferase gene, 1 kinase gene, 1 glucosyltransferase gene and 1 isomerase gene for encoding thuringiensin are confirmed, and the amino acid sequences encoded by those genes as shown in SEQ ID NO: 2-12 are also provided.

The present invention still provides the following genes in SEQ ID NO: 1: thu1 locates in No. 2010-2315 bases of the nucleotide sequence of the gene cluster with a length of 305 base-pairs encoding 101 amino acids and encoding polysaccharide polymerizing protein; thu2 locates in No. 5790-7475 bases of the nucleotide sequence of the gene cluster with a length of 1685 base-pairs encoding 561 amino acids and encoding domain of nonribosomal peptide/polyketide synthetase domain; thu3 locates in No. 10125-11060 bases of the nucleotide sequence of the gene cluster with a length of 935 base-pairs encoding 311 amino acids and encoding protein DUF894; thu4 locates in No. 12015-12446 bases of the nucleotide sequence of the gene cluster with a length of 431 base-pairs encoding 143 amino acids and encoding methyltransferase.

And the each encoding step of encoding the thuringiensin, post modifying process, adjusting and transporting process are corresponding to thuringiensin biosynthetases defined by SEQ ID NO: 2-12.

The invention still provides the approach of isolating the genes encoding thuringiensin from the recombinant vector carrying at least part of SEQ ID NO:1 or from microbial gene library.

The invention still provides the approaches of effectively isolating and purifying thuringiensin.

The invention still provides the approach of obtaining recombinant DNA vector which contains at least part of the nucleotide sequence as shown in SEQ ID NO:1.

The invention still provides the approach of transforming the recombinant DNA vector which contains at least part of the nucleotide sequence as shown in SEQ ID NO:1 into a host cell which does not produce thuringiensin.

The invention still provides the approach of obtaining microbial mutants in which the plasmids contain the interrupted genes encoding thuringiensin, and the genes of at least one of the mutants contain the nucleotide sequence as shown in SEQ ID NO:1.

The complementary sequence of SEQ ID NO:1 can be obtained any time based on the principle of complementary base pairing in DNA. The nucleotide sequence or part of the nucleotide sequence of SEQ ID NO:1 can be obtained any time by Polymerase Chain Reaction (PCR), digesting relative DNA with proper enzyme or employing other appropriate technology. The DNA fragments or genes containing one or more sequences according to the invention can be obtained any time. With the nucleotide sequences or part of the nucleotide sequences according to the invention, genes similar to the genes encoding thuringiensin can be obtained from other organisms through the measures of Polymerase Chain Reaction or conducting Southern blotting by using the DNA containing the sequences according to the invention as a probe.

The nucleotide fragments according to the invention can be used to isolate a domain with biological activity from Bacillus thuringiensis CT-43 strain or other strains. For example, a domain of nonribosomal peptide/polyketide synthetase and biological activity site of modifying gene can be obtained by Polymerase Chain Reaction, enzyme cutting site or other proper measures.

The nucleotide sequences according to the invention can fuse with nucleotide sequence of vector to obtain a recombinant sequence and the corresponding DNA molecules.

New thuringiensin derivatives can be obtained from the cloned genes or DNA fragments containing the nucleotide sequence or at least part of the nucleotide sequence according to the invention by interrupting one or more steps of encoding thuringiensin.

The cloned DNA containing the nucleotide sequence or at least part of the nucleotide sequences according to the invention can be used to locate more library plasmids from the genome library of Bacillus thuringiensis CT-43 strain. These library plasmids contain at least part of the nucleotide sequence and the uncloned DNA in the adjacent region before the nucleotide sequences.

The nucleotide sequences according to the invention can be modified or mutated by ways of insertion or replacement, Polymerase Chain Reaction, wrongly mediated Polymerase Chain Reaction, site specific mutation, recombination of different sequences, or induction by UV or chemical agents.

The nucleotide sequences according to the invention can conduct DNA shuffling with different parts of the sequences or homologous sequences from other origins.

The cloned gene containing the nucleotide sequences or part of the nucleotide sequences according to the invention can be expressed in a foreign host through suitable expression system, so as to obtain modified thuringiensin or thuringiensin with higher biological activity or higher thuringiensin yield. These foreign hosts include Escherichia Coli, Bacillus, yeasts, plants or animals, etc.

The modifying gene, transporter gene and glucosyltransferase gene which contain the nucleotide sequences or at least part of the nucleotide sequences according to the invention can be used to construct a derived library or combinatorial library.

One or more fragments containing the nucleotide sequences or at least part of the nucleotide sequences according to the invention can be cloned into a modified bacteria artificial chromosome (BAC), yeast artificial chromosome (YAC), cosmid, expression vector or other kinds of vectors to meet the appropriate requirements.

The genes or gene cluster containing the nucleotide sequences or at least part of the nucleotide sequences according to the invention can be expressed in a foreign host, and their roles in metabolic chain of the host can be understood by proteomics technology.

The polypeptides containing the amino acid sequences or at least part of the amino acid sequences as shown in SEQ ID NO: 2-12 according to the invention may still have biological activity or even new biological activity, an increased yield, optimized protein dynamics properties, or other desired properties.

New proteins or enzymes can be obtained by connecting the amino acid sequences as shown in SEQ ID NO: 2-12 with suitable technical deletion, thereby to generate new related products.

The present invention has substantive features and represents notable progress. The nucleotide sequence as shown in SEQ ID NO: 1 according to the invention can be used to generate genetic engineering insecticidal elements or increase the yield of the genetic engineering insecticidal elements. The amino acid sequences as shown in SEQ ID NO: 2-12 according to the invention can be used to isolate the desired proteins and prepare antibodies. The amino acid sequences as shown in SEQ ID NO: 2-12 according to the invention provide the possibility of predicting the three-dimensional structure of nonribosomal peptide/polyketide synthetase, and provide foundation of modifying or improving protein activity. The genes and the proteins encoded by the genes according to the invention, the corresponding antibodies or nucleosides can be used to search and develop the compounds or proteins applying in medicine, industry and agriculture.

### DESCRIPTION OF DRAWINGS

SEQ ID NO: 1 in the sequence listing is the nucleotide sequence of the gene cluster encoding thuringiensin according to the invention;

SEQ ID NO: 2-12 in the sequence listings are the amino acid sequences of thuringiensin encoded by the gene cluster according to the invention;

FIG. 1 is the structure of the whole gene cluster of thuABCDEFG encoding the thuringiensin biosynthetase. The gene cluster includes structural gene, modifying gene, transporting gene and assembled gene, encoding and transporting the thuringiensin biosynthetase;

FIG. 2 is a schematic figure of the chemical structure of thuringiensin;

FIG. 3 is a mass spectrogram of thuringiensin, and the molecular weight of thuringiensin is 701;

FIG. 4 is a diagram of plasmid pBMB0558 containing cry1B gene;

FIG. 5A is a HPLC detection diagram of thuringiensin of the supernatant from Bacillus thuringiensis CT-43 strain;

FIG. 5B is a HPLC detection diagram of thuringiensin of the supernatant from heterologous expression Bacillus thuringiensis BMB0542 strain;

FIG. 6 is a diagram of the approach of encoding thuringiensin;

FIG. 7 is a diagram of the approach of assembling thuringiensin.

### SPECIFIC MODE FOR CARRYING OUT THE INVENTION

### Example 1

### Locating the Gene Cluster Synthesizing (Encoding) Thuringiensin

1. Screening Plasmid-Cured Mutant of Bacillus thuringiensis CT-43 Strain

Bacillus thuringiensis CT-43 strain came from Bacillus thuringiensis stain published by the applicant (SUN Ming & YU Ziniu, Characteristics of Parasporal Crystal Protein from Bacillus Thuringiensis Subsp. Chinensis CT-43 strain, Acta Microbiologica Sinica, 1996, 36: 303-306), eliminating plasmid by step-up increasing temperature, particularly including the following steps: 6 plasmid-cured mutant strains in total are obtained, and respectively named as CT-43-1c, CT-43-7, CT-43-5, CT-43-55, CT-43-62 and BMB0806 (DONG Chunming et al., Screening and Characterization of a Thuringiensin Mutant from Bacillus thuringiensis, Chinese Journal of Applied & Environmental Biology, 2007, 13: 526-529), wherein CT-43-1c and CT-43-7 had lost the 130 kDa and 65 kDa insecticidal crystal proteins, but they still can generate thuringiensin; CT-43-5, CT-43-55 and CT-43-62 had lost 140 kDa insecticidal crystal protein, and can not generate thuringiensin; BMB0806 is acrystalliferous mutant which does not generate thuringiensin; it can be seen that, the 140 kDa insecticidal crystal protein in CT-43 strain is probably connected with producing thuringiensin.

The specific operation method for plasmid elimination includes steps of: streaking LB (1% peptone; 0.5% yeast powder; 1% NaCl; pH 7.0-7.2) plate to separate the single colonies of Bacillus thuringiensis CT-43 strain thereon; inoculating single colony in LB liquid medium, culturing at 30 until the middle of logarithmic growth phase, re-inoculating with inoculation amount of 1/100 (v/v) in SCG (0.1% Vatamin free casamino acids; 0.5% glucose; 0.2% (NH₄)₂SO₄; 1.4% K₂HPO₄; 0.5% KH₂PO₄; 0.1% sodium citrate; 0.02% MgSO₄; pH 7.0-7.2) liquid medium, culturing at 42 with rotational speed of 200 r/min, re-inoculating for every 12 hours, conducting gradient dilution after continually re-inoculating for 10 times, then spreading the SCG plate and culturing at 42; getting single colony from the above SCG plate and inoculating on SCG plate by spot inoculation, and culturing in incubator at 42; getting edge part of the colony on the above SCG plate and inoculating on the next SCG plate by spot inoculation for every 48 hours, meanwhile, extracting the plasmid and detecting plasmid belts by electrophoresis; inoculating part of the strains treated at 42 on SCG plate by spot inoculation and culturing with the temperature increased to 44, getting edge part of the colony on the above SCG plate and inoculating on the next SCG plate by spot inoculation for every 48 hours, meanwhile, extracting the plasmid and detecting plasmid belts by electrophoresis.

2. The Distribution of cry1B Gene in the Plasmid-Cured Mutant of CT-43 strain

Conducting detection experiment on whether cry1B gene exists in Bacillus thuringiensis CT-43 strain and its mutant, the result shows the high thuringiensin yield phenotype of the strain is directly relative to the existence of cry1B gene. Taking the total DNA of the CT-43 strain and its mutant as template to amplify with specific primer of cry1B gene, the result shows wild CT-43 strain, CT-43-1c mutant and CT-43-7 strain have cry1B gene belts after amplification, while results of amplification about CT-43-5 mutant, CT-43-55 and CT-43-62 strains are negative. It is inferred that cry1B gene is located on an endogenous big plasmid in wild CT-43 strain, and the plasmid is closely related to encoding thuringiensin.

cry1B-1 and cry1B-2 are amplified by PCR amplification which are designed based on the sequence of cry1B gene (NCBI No: X06711 originated from Bt thuringiensis HD-2), and the DNA sequences of the primer pairs are as follows:

cry1B-1: 5′-CTTCATCACGATGGAGTA-3′
cry1B-2: 5′-CATAATTTGGTCGTTCTG-3′

PCR reaction system: 2 μl of 10× buffer, 1.5 μl of 2 mmol/L dNTP, 0.4 μl of each 10 μmol/L primer, 1 U of Taq enzyme, 1 μl of total genome DNA of CT-43, adding sterile deionized water to a total volume of 200.

PCR amplification procedure: step 1, pre-degeneration at 94 for 5 min; step 2, degeneration at 94° C. for 1 min; step 3, renaturation at 55° C. for 1 min; step 4, extension at 74° C. for 1.5 min; step 5, turning to step 2 and going on 28 cycles; step 6, extension at 72° C. for 5 min.

3. Locating the Big Plasmid Carrying cry1B Gene

Extracting Bacillus thuringiensis CT-43 strain and its mutant and HD-2 plasmid, and taking the purified PCR product of cry1B gene as probe to conduct DIG-Labeled Southern blotting experiment, the result shows the big plasmids of wild CT-43 strain, CT-43-1c mutant and CT-43-7 strain which contain cry1B genes have high degree of homology with the 75 MDa big plasmid of HD-2, while there is no hybridization signs for CT-43-5, CT-43-55, CT-43-62 and BMB0806 strains, combining with the above circumstance of producing thuringiensin, the gene related to encoding thuringiensin is initially located on the big plasmid containing cry1B gene, and the big plasmid is named pBMB0558.

The procedure of extracting Bacillus thuringiensis plasmid comprising the following steps:

(1) activating Bacillus thuringiensis CT-43 strain overnight, re-inoculating with inoculation amount of 1/100 (v/v) in 5 mL fresh LB liquid medium, conducting shaking culture at 30° C. with a rotational speed of 200 r/min until the middle of logarithmic growth phase;

(2) collecting all the bacteria by centrifugation (12000 r/min, 30 sec), and washing the bacteria one time with STE (100 mmol/L NaCl; 10 mmol/L Tris.HC1 [pH 8.0]; 1 mmol/L EDTA);

(3) adding 200 μL solution I (50 mmol/L glucose; 25 mmol/L Tris.HC1 [pII 8.0]; 10 mmol/L EDTA) to make bacteria suspension, then adding 20 μL of 50 mg/mL lysozyme; putting it into ice bath over 1 hour;

(4) adding 400 μL of newly prepared solution II (0.2 mol/L NaOH; 1% sodium dodecyl sulfate (SDS) and mixing gently, then putting it into ice bath for 5-10 min;

(5) adding 300 μL of solution III (60 mL of 50 mol/L KAc; 11.5 mL of glacial acetic acid; 28.5 mL of deionized water), putting it into ice bath for 5 min after mixing uniformly;

(6) centrifugating at a rotational speed of 12000 r/min for 5 min, sucking the supernatant into a new Eppendorf tube, extracting with phenol/chloroform/isopentanol solution (25:24:1; v/v/v) for 2 times;

(7) adding 2 times volume of dehydrated ethanol or the same volume of isopropanol into the supernatant, standing for 5-10 min at room temperature, centrifugating at a rotational speed of 12000 r/min for 5 min, then removing the supernatant and washing the sediment one time with 70% ethanol by centrifugation;

(8) drying the sediment under vacuum condition, dissolving the sediment with 10-30 μL of TE solution (1 mmol/L EDTA; 10 mmol/L Tris.HC1 [pH 8.0]), then adding 1-2 μL of 10 mg/mL RNase solution, standing at 37 over 0.5 h after mixing uniformly, and the resulting mixture is the prepared DNA solution.

The plasmids of Bacillus thuringiensis CT-43 strain and its mutants CT-43-1c, CT-43-7, CT-43-5, CT-43-55, CT-43-62, BMB0806 and strain HD-2 (Levinson B L, Kasyan K J, Chiu S S, Currier T C, Gonzalez J M. Identification of beta-exotoxin production, plasmids encoding beta-exotoxin, and a new exotoxin in Bacillus thuringiensis by using high-performance liquid chromatography. J Bacterial, 1990, 172: 3172-3179) were extracted through the above process, the chromosome and different sizes of plasmids were isolated by conventional electrophoresis (0.8% gel; electrophoresis time 4-5 hours; 125V; 4), and transferred on the positively charged nylon membrane by the capillary tube method. cry1B gene was amplified with primers cry1B-1/cry1B-2 in CT-43 strain, and the probe was labeled by digoxin. The detailed operating procedure see: MOLECULAR CLONING: A LABORATORY MANUAL; third edition, by Joseph Sambrook et al, Chinese version, 2002, Science Press, Beijing.

### Example 2

### Cloning Large Plasmid pBMB0558 Containing cry1B Gene

1. Construction of Bacillus thuringiensis Strain CT-43 Genomic BAC Library

(1) Preparation of Exogenous DNA Fragments

Single colony of strain CT-43 was picked and incubated in 5 mL of sterilized LB medium, culturing at 28° C. overnight. Next day the cultured strain was re-inoculated with inoculation amount of 1% into 100 mL of sterilized LB medium prepared with deionized water next day, culturing at 28° C. for 3-4 h to make the OD₆₀₀ reach 0.2. The bacteria were collected in a clean sterile 50 mL centrifugal tube and centrifuged with a rotation speed of 10,000 rpm, washed 2 times with TE buffer in the same volume as the culture medium. 1% low melting agarose gel was prepared with TE25S buffer and cooled to 50° C., and the bacteria were resuspended in 2 mL of gel. The gel bacterial suspension was diluted with gel at 50° C. by 2-fold, 4-fold, 6-fold, 8-fold, 10-fold, respectively. Each dilution was poured into a mold to prepare the bacteria gel-embedded block.

The embedded block was immersed in TE25S buffer, and lysozyme with a final concentration of 2 mg/mL was added thereto, treating at 4° C. for 24 h to remove the cell wall. The TE25S buffer was removed, and then the embedded block with NDS was washed 3 times in total, 10 mL for each time. The embedded block was immersed in NDS buffer (0.5 M EDTA, 100 mM Tris-Cl, 1% SDS, pH 8.0), and proteinase K with a final concentration of 1 mg/mL was added thereto, placed into a 50° C. water bath for 48 h to remove the protein. The NDS buffer was removed, washed three times with common T₁₀E₁₀ buffer (10 mM Tris-Cl, 10 mM EDTA, pH 8.0, 121° C., 30 min sterilization), and then the embedded block was transferred into another 50 mL centrifuge tube, washed five times with T₁₀E₁₀ buffer containing 0.1 mM PMSF, 10 mL for each time for 1 h and operated on the ice. The washed embedded block can be directly digested, or stored at 4° C. for more than 7 days, or immersed in 70% ethanol at −20° C. for more than 2 months.

The DNA content of the embedded blocks was detected by pulse field gel electrophoresis (PFGE) (1% gel; starting pulse: 1; termination pulse: 25; voltage: 6 V/cm; conversion angle: 120°; and electrophoresis time: 24 h), the appropriate dilution of embedded blocks were selected. 1-fold Buffer M was prepared with sterile deionized water, and each embedded block was immersed with 500 μL 1-fold Buffer M, and then placed on the ice for 2 h. The 1-fold Buffer M was removed, then fresh 1-fold Buffer M was added, 500 μL of enzyme digestion system was set for each embedded block with HindIII enzyme amount of 0.2 U, 0.5 U, 1 U, 2 U, 5 U, 10 U, respectively, followed by putting them on the ice for 30 min and incubating at 37° C. for 30 min 0.5 M EDTA was used to terminate reaction after digestion. The size of the digestion products was detected by PFGE, the swimming positions of the reaction system and the target fragments (75-100 kb) were determined. The reaction system was amplified, followed by conducting pulsed electrophoresis, and cutting the target fragments in the corresponding location of the gels. The target DNA was recovered from the gel containing target fragment through the dialysis bags electrophoresis (TAE buffer; voltage: 6 V/cm; electrophoresis time: 2 h), stored at −20° C. for later use.

(2) Preparation of BAC Carrier

E. coli containing the BAC plasmid was inoculated in LB medium, and chloramphenicol with a final concentration of 30 ng/mL was added thereto, cultured at 37° C. overnight. The bacteria were collected, and then plasmids were extracted by the low alkali method. The extracted BAC carrier was completely digested by the restriction enzyme HindIII. The digested reaction system was treated at 65° C. for 10 min to inactive the enzyme. The alkaline phosphatase was added to the existing system in an enzyme amount of 5 U CIAP per 1 μg of plasmid, incubated at 37° C. for 1 h, the reaction was terminated by sample buffer with a final concentration of 1-fold.

The dephosphorylated carrier was detected by PFGE (1% gel; starting pulse: 1; termination pulse: 15; voltage: 4.5 V/cm; conversion angle: 120°; electrophoresis time: 18 h). Only part of the gel was cut and stained by ethidium bromide (EB), a clean-cut blade was used to engrave on the geland the position of the carrier (11.4 kb) was marked. The gel containing carrier in the corresponding position without stained was cut off, and the carrier was recovered through the dialysis bags electrophoresis (TAE buffer; voltage: 6 V/cm; electrophoresis time: 2 h). Ligation system was set for the recovered carrier in the amount of 1 U T4 DNA ligase per 1 μg DNA, ligation was conducted at 16 overnight. The sample was separated by pulse electrophoresis, and the gel containing the carrier (11.4 kb linear DNA) was cut off, followed by recovering by dialysis bags electrophoresis and storing at −20 for later use.

(3) Preparation of Electroporation-Competent Cells of E. coli

E. coli strain DH10B single colony was inoculated in 5 mL of SOB medium (medium per liter contains 20 g of peptone, 5 g of yeast powder, 0.5 g of NaCl, 250 in M of KCl and 10 mM of MgCl₂; Luo M Z, Wing R A. An improved method for plant BAC library construction. In: Grotewold E eds., Methods in Molecular Biology. Totowa, N.J.: Humana Press, 2003), and cultured at 37 overnight. The inoculated DH10B strain was re-inoculated in 100 mL of SOB medium with an inoculation amount of 1%, cultured at 37 for 3 h to make the OD₆₀₀ value reach 0.6. The culture medium was rapidly cooled on ice, and then centrifuged at 4000 rpm at 4 for 10 min to collect bacteria. The bacteria were washed with 10% pre-cooling glycerol for three times, the same volume of glycerol as the medium was used for the first time, and the volume of glycerol half of the medium was used for the rest of two times. The bacteria were resuspended in 2 mL of 10% pre-cooling glycerol and distributed into 50 μL, per tube, frozen immediately and stored at −70 for later use. These operations should be carried out under strict sterile conditions at 4 in thermostatic room. Competent cells were spread on LB plates containing chloramphenicol with a final concentration of 12.5 μg/mL to test whether the cells were contaminated.

(4) Ligation and Transformation

The λ DNA HindIII fragments were ligated with the prepared BAC carrier, and the prepared E. coli strains DH10B competent cells were transformed by electroporation (voltage 12.5 kv/cm), then the efficiency of carriers and competent cells were tested. The ligation system was set according to foreign sources and carrier in a molar ratio of 10:1, followed by connecting at 16 overnight. 1% gel containing 0.1 M glucose was prepared with deionized water, followed by melting the gel and preparing the gel column with grooves with a capacity of 50 μL liquid. The ligation system was put in the grooves, followed by keeping at 4 for 2 h to remove the salt from the ligation system. 2 μL of connected products was mixed with 50 μL of competent cells, followed by transforming by electroporation (voltage 2.5 kv/cm), then quickly adding preheated SOC medium (100 ml SOB medium supplemented with 20 ml of 1M glucose; Luo M Z, Wing R A. An improved method for plant BAC library construction. In: Grotewold E eds., Methods in Molecular Biology. Totowa, N.J.: Humana Press, 2003) thereto, conducting restoration culture at 37 for 40 min. X-gal and IPTG were spread on the surface of LB plates containing 12.5 μg/mL chloramphenicol (each plate dish has a diameter of 9 cm), and the restoration culture medium was spread on the plate, cultured at 37 for 16 h. Single white colonies were picked and stored.

(5) Library Screening and Preservation

Single white colonies were randomly picked and inoculated in 5 mL of LB medium, chloramphenicol with a final concentration of 12.5 μg/mL was added thereto, cultured at 37 overnight. The bacteria were collected, followed by extracting plasmid by the low alkali method. BAC transformants were digested with restriction enzymes NotI and HindIII respectively, and the length of the insert fragment was detected by pulse electrophoresis. A large quantity of transformants from the batches with appropriate inserting fragment size and high positive rate were picked and inoculated in LB medium containing 20% glycerol and 12.5 μg/mL chloramphenicol, statically cultured with 96 cell culture plate (200 μL medium/well) for 24 h. The cultured bacterial suspension was frozen immediately in liquid nitrogen, and then stored at −80.

2. Construction of pBMB0558 Overlapping Linkage Groups

The selected single BAC clones containing cry1B gene was subjected to single digestion and double digestion by BamHI and NotI, and the digested products were subjected to pulse electrophoresis, followed by drawing the overlapping linkage groups map by software based on the digested fragment size and location of each other in the pulse electrophoresis figure.

3. PBMB0558 Sequencing

Based on the overlapping linkage map, two BAC clones sufficient to cover the entire pBMB0558 plasmid were selected to construct and sequence subclone library.

4. PBMB0558 Sequence Splicing and Bioinformatic Analysis

The sequence was spliced by DNAStar 7.0, and the gap was filled by PCR. Comparing and analyzing through the GenBank database, it was found that an approximately 12 kb gene cluster exist in the plasmid, and the gene cluster was named thuABCDEFG.

### Example 3

### Heterologous Expression of thuABCDEFG Gene Cluster

1. Heterologous Expression of BAC Clones Containing thuABCDEFG Gene Cluster

BAC clones pBMB0542 (inserting external sources of about 23 kb) containing thuABCDEFG gene cluster were transferred into Bacillus thuringiensis plasmid-free mutant BMB171 bp electroporation (LI Lin, YANG Chao, LIU Ziduo, LI Fudi, YU Ziniu, Screening of Acrystalliferous Mutants from Bacillus Thuringiensis and Their Transformation Properties, Acta Microbiologica Sinica, 2000, 40: 85-90), and the production of thuringiensin were verified by HPLC and MS, the results showed that the yield of thuringiensin was approximately one third of that of wild Bacillus thuringiensis strain CT-43.

Recombinant Bacillus thuringiensis BMB0542 capable of producing thuringiensin obtained in this invention had been deposited in China Center for Type Culture Collection (CCTCC) in Wuhan University, Wuhan, Hubei, on Jan. 8, 2009, accession number: CCTCC NO M209011.

2. Separation Method for Purifying Thuringiensin (Extracting with Organic Solvent):

(1) Single colony of Bacillus thuringiensis strain CT-43 producing thuringiensin was inoculated in 5 mL of LB culture medium, cultured at 30 at 200 rpm overnight.

(2) The bacteria were re-inoculated in 25 mL of LB culture medium at 30 with inoculation amount of 1%, cultured continuously for 24 hours, at 200 rpm.

(3) 100 μL of the fermentation supernatant was added into 900 μL of acetone to achieve a final concentration of 90%, followed by mixing uniformly and centrifuging at 12000 rpm for 6 min.

(4) The precipitate obtained by centrifugation was re-dissolved in 100 μL of deionized water, followed by adding 67 μL of acetonitrile to achieve a final concentration of 40%, then mixing uniformly and centrifuging at 12000 rpm for 6 min.

(5) The white precipitate obtained by centrifugation was removed, and acetonitrile was added into the supernatant again to achieve a final concentration of 90%, centrifuged for 6 min at 12000 rpm, the resulting precipitate was collected by centrifugation and dried with brown color.

(6) The precipitate was dissolved in 25-50 μL of mobile phase, filtering with a membrane, 20 μL of the filtrate was sampled for HPLC.

High pressure liquid chromatography (HPLC) method: the purified thuringiensin or the sample was assayed on WATERS 2487 HPLC. Chromatographic column: Agilent C-18 (25 cm×4.6 mm, 5 μm); injection volume: 20 μL; detection wavelength 260 nm; mobile phase: 50 mM KH₂PO₄ and 5% methanol (pH was adjusted to 3.0 with phosphoric acid); flow rate: 1 mL/min; retention time of thuringiensin: about 8.0 min.

3. Bacterium Characteristics and Genetic Characteristics of the Recombinant Bacillus thuringiensis Strain BMB0542:

Biological characteristics: the bacterium is straight rhabditiform, the trophozoite is chain-typed or single, gram-positive, the spore is cylindrical or nearly oval, one-sided, the spore capsule does not expand, colonies show round shape in beef extract peptone medium with smooth edges, the wax-like lawn is full; the growth temperature is 10-45, the optimum growth temperature is 26-32; the suitable pH is 6.8-7.4, facultative anaerobic; when growing in 1% NaCl beef extraction peptone medium the parasporal crystal is in shape of diamond. In the fermentation, the yield of thuringiensin in the supernatant reaches the highest when the incubation time reaches up to 24 hours.

Genetic characteristics: the genetical engineering bacteria of the invention is obtained from the natural Bacillus thuringiensis CT-43 as a parent strain, it contains gene cluster capable of synthesizing thuringiensin. After subculture, each gene in the engineering strain is stable and shows normal expression without significant impact on the growth of recipient bacteria.

### Example 4

### Approach of Encoding and Assembling Thuringiensin

1. Bioinformatics Analysis of thuABCDEFG Gene Cluster

In thuABCDEFG, the protein encoded by thuA shows 31% amino acid sequence identity to 6-phosphogluconate-1-dehydrogenase; the protein encoded by thuB shows 36% amino acid sequence identity to aspartic acid, glutamic acid, hydantoin helicase family protein in Roseovarius sp. HTCC2601; the protein encoded by thuC shows 97% amino acid sequence identity to phosphotransferase in Shigella sonnei Sd197; the protein encoded by thuD shows 73% amino acid sequence identity to ribose dehydrogenase in Bacillus halodurans C-125, the enzyme belonges to uridine diphosphate (UDP)-glucose dehydrogenase family, mainly catalyzes the oxidation of the alcohols depending on nicotinamide adenine dinucleotide (NAD) to transform into acids; thuE protein shows 31% amino acid sequence identity to shikimic acid kinase in Streptococcus pyogenes MGAS10394; thuF protein shows 43% amino acid sequence identity to glycosidic transferase in Streptococcus pyogenes MGAS 10394, the enzyme transfers sugars to a series of substrates such as cellulose, phosphate dolichol and teichoicteichoic-acid; thuG protein shows 98% amino acid sequence identity to N-acetyl-D-glucosamine-2-isomerase in the Escherichia coli B 171, mainly mediates the isomerization in the N-acetyl neuraminic acid biological encoding, with the catalytic mechanism of addition and subtraction effect of nucleotides regulated by ATP.

In the gene cluster, in addition to thuA to thuG, there are four ORFs. Therein, the protein encoded by thu1 shows 32% amino acid sequence identity to polysaccharide polymerization protein of Rhodopseudomonas palustris CGA009; the thu2 protein shows 35% amino acid sequence identity to nonribosomal peptide-encoding enzyme of Myxococcus xanthus DK 1622; the thu3 protein shows 36% amino acid sequence identity to macrolide circulating protein of Streptococcus pyogenes MGAS10394; the thu4 protein shows 55% amino acid sequence identity to transmethylase dependent on S-adenosylmethionine of Salmonella enterica subsp. enterica serovar Javiana str.

### GA_MM04042433.

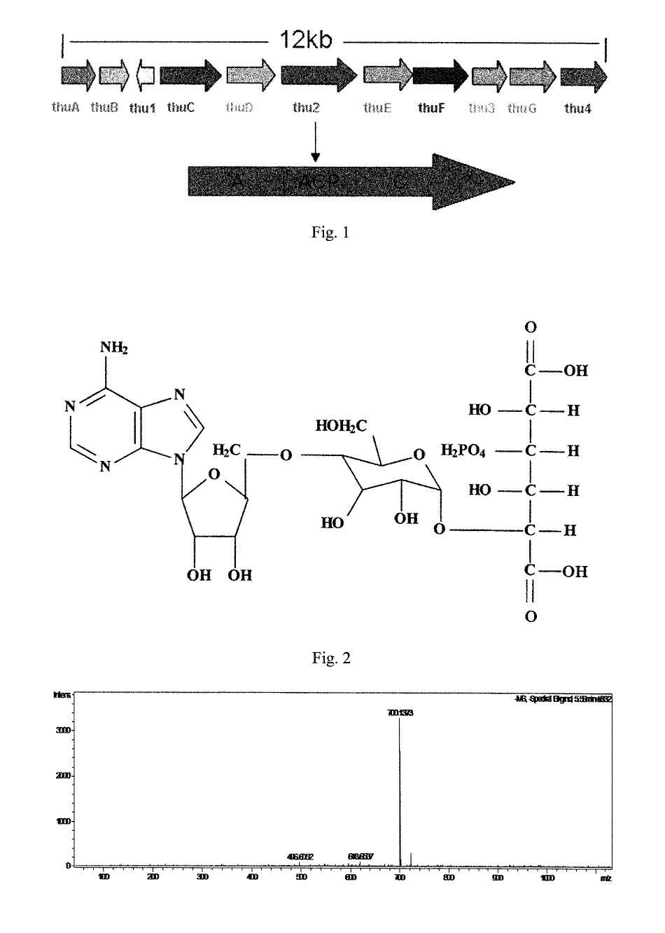

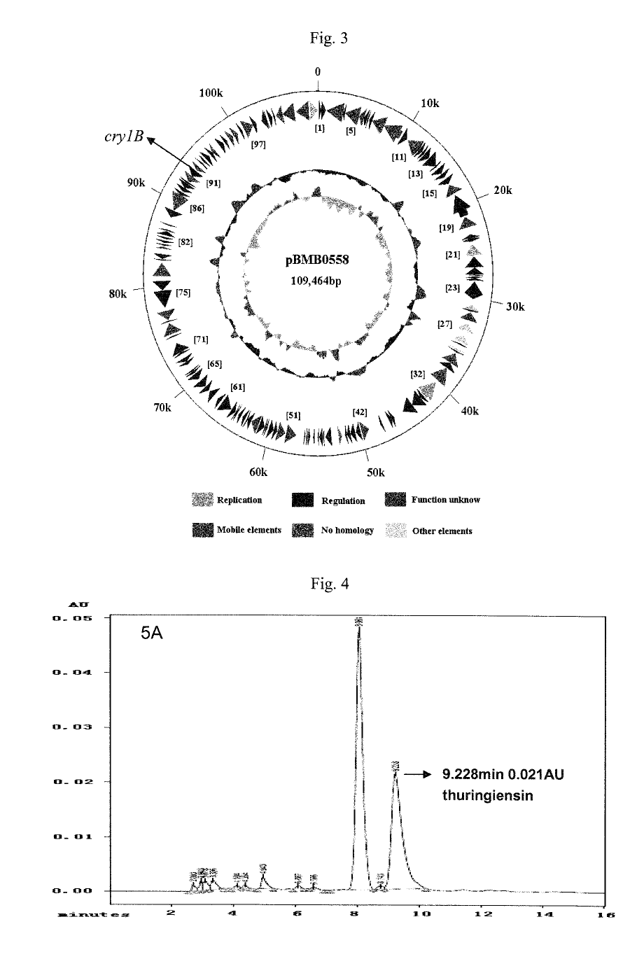

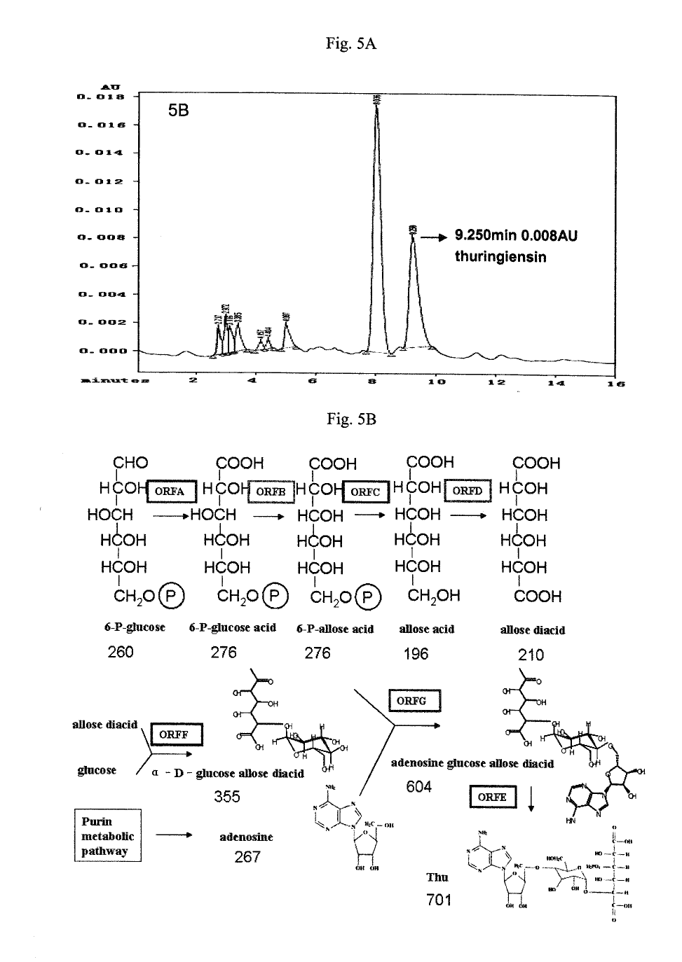

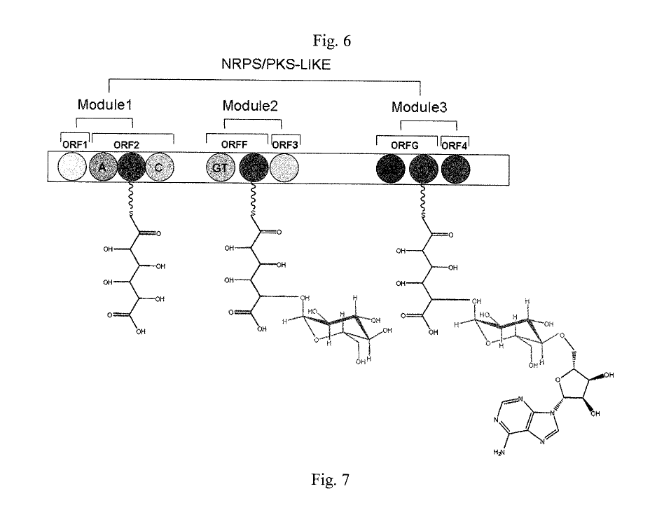

Shown in FIG. 1, the sequences in the sequence listings according to the invention are described as follows:

SEQ ID NO: 1 is a nucleotide sequence of 12,446 bp including 11 open reading frames, which are gene thuA, thuB, thuC, thuD, thuE, thuF, thuG, thu1, thu2, thu3 and thu4 for biologically encoding thuringiensin.

SEQ ID NO: 2 is an amino acid sequence of glucose-6-phosphate dehydrogenase encoded by thuA gene (nucleotide 1-363 in SEQ ID NO: 1).

SEQ ID NO: 3 is an amino acid sequence of helicase encoded by thuB gene (nucleotide 364-681 in SEQ ID NO: 1).

SEQ ID NO: 4 is an amino acid sequence of phosphate transferase encoded by thuC gene (nucleotide 2617-4344 in SEQ ID NO: 1).

SEQ ID NO: 5 is an amino acid sequence of UDP-glucose dehydrogenase encoded by thuD gene (nucleotide 4661-5278 in SEQ ID NO: 1).

SEQ ID NO: 6 is an amino acid sequence of shikimate kinase encoded by thuE gene (nucleotide 7987-8673 in SEQ ID NO: 1).

SEQ ID NO: 7 is an amino acid sequence of glucosyltransferase encoded by thuF gene (nucleotide 8670-9798 in SEQ ID NO: 1).

SEQ ID NO: 8 is an amino acid sequence of glucosamine isomerase encoded by thuG gene (nucleotide 10957-11637 in SEQ ID NO: 1).

SEQ ID NO: 9 is an amino acid sequence of polysaccharide polymerization protein encoded by thu1 gene (nucleotide 2010-2315 in SEQ ID NO: 1).

SEQ ID NO: 10 is an amino acid sequence of nonribosomal peptide/polyketide coding enzyme domain encoded by thu2 gene (nucleotide 5790-7475 in SEQ ID NO: 1).

SEQ ID NO: 11 is an amino acid sequence of DUF894 encoded by thu3 gene (nucleotide 10125-11060 in SEQ ID NO: 1).

SEQ ID NO: 12 is an amino acid sequence of methyltransferase encoded by thu4 gene (nucleotide 12015-12446 in SEQ ID NO: 1).

Further detailed description of the invention is as follows:

Since thuringiensin has the broad spectrum insecticidal activity, it has different degrees of activity against Lepidoptera, Diptera, Orthoptera, Coleoptera, Hymenoptera, Hemiptera, Isoptera etc. and it also has higher insecticidal activity against the aphids, nematodes and mites, particularly nematodes. In the invention, the gene cluster encoding thuringiensin was cloned; through constructing the genome and BAC library of Bacillus thuringiensis CT-43, large plasmid pBMB0558 containing gene cluster of thuringiensin was cloned, the continuous nucleotide sequence of 109,464 bp was sequenced, wherein, 12,446 bp belongs to nucleotide sequence of gene cluster encoding thuringiensin. The sequence analysis was accomplished by Clone 5 software and Conserved Domain Database search of the US National Center for Biotechnology Information and the worldwide Blast engine thereof.

Bases 1-363, 364-681, 2617-4344 and 4661-5278 in SEQ ID NO: 1 are the gene sequences encoding allomucic acid which is a precursor of thuringiensin. The nucleotide sequences and corresponding amino acid sequences of the genes are shown in Table 1:

TABLE 1
The nucleotide sequences and the amino acid sequences encoding
allomucicacid which is a precursor of thuringiensin

| | Base position | Corresponding |
|---|---|---|
| Gene | in SEQ ID NO: 1 | amino acid sequence |
| thuA | 1-363 | SEQ ID NO: 2 |
| thuB | 364-681 | SEQ ID NO: 3 |
| thuC | 2617-4344 | SEQ ID NO: 4 |
| thuD | 4661-5278 | SEQ ID NO: 5 |

Bases 7987-8673, 8670-9797 and 10957-11637 in SEQ ID NO: 1 are the gene sequences assembling thuringiensin precursor. The nucleotide sequences and corresponding amino acid sequences are shown in Table 2:

TABLE 2
The nucleotide sequences and the amino acid sequences assembling
thuringiensin precursor

| | Base position | Corresponding |
|---|---|---|
| Gene | in SEQ ID NO: 1 | amino acid sequence |
| thuE | 7987-8673 | SEQ ID NO: 6 |
| thuF | 8670-9797 | SEQ ID NO: 7 |
| thuG | 10957-11637 | SEQ ID NO: 8 |

Bases 2010-2315, 5790-7475 and 10125-11060 in SEQ ID NO: 1 are the gene sequences for the suspension and extension of thuringiensin precursor. The nucleotide sequences and corresponding amino acid sequences were shown in Table 3:

TABLE 3
The nucleotide sequences and the amino acid sequences for the
suspension and extension of thuringiensin precursor

| | Base position | Corresponding |
|---|---|---|
| Gene | in SEQ ID NO: 1 | amino acid sequence |
| thu1 | 2010-2315 | SEQ ID NO: 9 |
| thu2 | 5790-7475 | SEQ ID NO: 10 |
| thu3 | 10125-11060 | SEQ ID NO: 11 |

Base 12015-12446 in SEQ ID NO: 1 is the gene sequence for the modification after encoding thuringiensin, and its nucleotide sequence and corresponding amino acid sequence is shown in Table 4:

TABLE 4
The nucleotide sequence and the amino acid sequence for the
modification after encoding thuringiensin

| | Base position | Corresponding |
|---|---|---|
| Gene | in SEQ ID NO: 1 | amino acid sequence |
| thu4 | 12015-12446 | SEQ ID NO: 12 |

The positions of domain of the suspension and extension module 1 in Thu2 are shown in Table 5:

TABLE 5
Domain of suspension and extension module 1

| | Amino acid position in Thu2 |
|---|---|
| Domain | (SEQ ID NO: 10) |
| A | 1-188 |
| C | 189-270 |
| ACP | 271-701 |

The functions of the genes in gene cluster encoding thuringiensin from Bacillus thuringiensis CT-43 are shown in Table 6:

TABLE 6
The functions of the genes in gene cluster encoding thuringiensin

| Gene | Production | Function |
|---|---|---|
| thuA | Glucose-6-phosphate-1- | Oxidizing aldehydes into |
| | dehydrogenase | carboxylic acid |
| thuB | Helicase | Mutarotation of isomer |
| thuC | Phosphate transferase | Transferring phosphate groups |
| thuD | UDP-glucose dehydrogenase | Oxidizing monobasic acid into |
| | | dibasic acid |
| thuE | Shikimate kinase | Adding phosphate groups |
| thuF | Glucosyltransferase | Adding glucose |
| thuG | Glucosamine isomerase | Adding adenosine |
| thu1 | Polysaccharide polymerization | Polymerizing polysaccharide |
| | protein | |
| thu2 | Acyl carrier protein | Assembling precursor |
| thu3 | DUF894 | Unknown function |
| thu4 | Methyltransferase | Modification |

The application examples illustrate the approach of obtaining and applying the sequences and elements according to the invention. These Examples are only used for illustration and not limiting the scope of the application of this invention. The derived genetic engineering strain with the changed sequence can be obtained by genetic engineering molecular operation to some gene sequences of the gene cluster, or the DNA fragments containing the sequence encoding thuringiensin can be obtained through the invention.

2. Approach of Encoding and Assembling Thuringiensin

The approach of encoding thuringiensin was obtained according to the above experimental data and comprehensive bioinformatics analysis. The approach mainly includes the process of encoding allomucic acid and assembling thuringiensin. The assembling process is similar to the nonribosomal peptide/polyketide coding approach in the antibiotics encoding approach, which requires a special acyl carrier protein. However, the assembly of thuringiensin is different from the reported nonribosomal peptide coding approach, polyketide coding approach or nonribosomal peptide/polyketide hybrid approach, the ACP proteins in the functional domain come from polyketide coding approach, while adenosine acylation domain (A-domain) and the condensation region (C-domain) are from nonribosomal peptide coding approach, thus the process of assembling thuringiensin is a similar nonribosomal peptide/polyketide coding approach integrated the functional modules of nonribosomal peptide and polyketide coding approach.

In this approach, different from the extending direction of chain of the conventional antibiotics, only the allomucic acid is suspended on the acyl carrier protein, then the allomucic acid is subjected to glycosylation reaction under the action of various enzymes, and adenosine was added continuously to form thuringiensin precursors without adding phosphate groups i.e. adenosine glucose allomucic acid. Methyltransferase in the gene cluster might also play a certain modification in the approach of assembling thuringiensin, so as to prevent the encoded precursor from being degraded by various enzymes in the cells.

1. A gene cluster from Bacillus thuringiensis capable of synthesizing thuringiensin, which comprises 11 genes of thuA, thuB, thuC, thuD, thuE, thuF, thuG, thu1, thu2, thu3 and thu4 with the nucleotide sequences as shown in SEQ ID NO: 1, and the thuringiensin biosynthetases encoded by the genes have the amino acid sequences as shown in SEQ ID NO: 2-12, the orders and positions of the genes in the cluster are as follows, respectively:
thuA locates in No. 1-363 bases of the nucleotide sequence of the gene cluster with a length of 363 base-pairs, which encodes 120 amino acids and encodes glucose-6-phosphate dehydrogenase;
thuB locates in No. 364-681 bases of the nucleotide sequence of the gene cluster with a length of 317 base-pairs, which encodes 105 amino acids and encodes helicase;
thuC locates in No. 2617-4344 bases of the nucleotide sequence of the gene cluster with a length of 1727 base-pairs, which encodes 575 amino acids and encodes phosphate transferase;
thuD locates in No. 4661-5278 bases of the nucleotide sequence of the gene cluster with a length of 617 base-pairs, which encodes 205 amino acids and encodes UDP-glucose dehydrogenase;
thuE locates in No. 7987-8673 bases of the nucleotide sequence of the gene cluster with a length of 686 base-pairs, which encodes 228 amino acids and encodes Shikimate kinase;
thuF locates in No. 8670-9797 bases of the nucleotide sequence of the gene cluster with a length of 1127 base-pairs, which encodes 375 amino acids and encodes glucosyltransferase;
thuG locates in No. 10957-11637 bases of the nucleotide sequence of the gene cluster with a length of 680 base-pairs, which encodes 226 amino acids and encodes glucosamine isomerase;
in SEQ ID NO: 1:
thu1 locates in No. 2010-2315 bases of the nucleotide sequence of the gene cluster with a length of 305 base-pairs, which encodes 101 amino acids and encodes polysaccharide polymerizing protein;
thu2 locates in No. 5790-7475 bases of the nucleotide sequence of the gene cluster with a length of 1685 base-pairs, which encodes 561 amino acids and encodes nonribosomal peptide/polyketide synthetase domain;
thu3 locates in No. 10125-11060 bases of the nucleotide sequence of the gene cluster with a length of 935 base-pairs, which encodes 311 amino acids and encodes protein DUF 894;
thu4 locates in No. 12015-12446 bases of the nucleotide sequence of the gene cluster with a length of 431 base-pairs, which encodes 143 amino acids and encodes methyltransferase.

2. A recombinant Bacillus thuringiensis strain capable of producing thuringiensin, which is recombinant Bacillus thuringiensis strain BMB0542, deposited in China Center for Type Culture Collection (CCTCC) with the deposit number of CCTCC No. M209011.

3. Use of the gene cluster capable of synthesizing thuringiensin according to claim 1 in producing thuringiensin.

4. Use of the recombinant Bacillus thuringiensis strain according to claim 2 in producing thuringiensin.
